(19) United States
(12) Patent Application Publication
(10) Pub. No.: US 2019/0256487 A1
(43) Pub. Date: Aug. 22, 2019

(54) HYDROXYNORKETAMINE PRODRUGS

(71) Applicant: BioPharmaWorks, LLC, Groton, CT (US)

(72) Inventors: Robert A. Volkmann, Mystic, CT (US); Anthony Marfat, Mystic, CT (US)

(21) Appl. No.: 16/333,635

(22) PCT Filed: Sep. 15, 2017
(86) PCT No.: PCT/US2017/051704

§ 371 Date: Mar. 15, 2019

Related U.S. Application Data
(60) Provisional application No. 62/395,579, filed on Sep. 16, 2016.

(51) Int. Cl. C07D 317/38; A61P 25/00; A61P 25/04; C07C 225/22

(52) U.S. Cl. CPC C07D 317/38; C07C 225/22; A61P 25/04; A61P 25/00

(57) ABSTRACT

Disclosed are novel ketamine prodrugs, pharmaceutical preparations containing such prodrugs of ketamine and methods of treating, bipolar depression, neuropathic and chronic pain, including complex regional pain synthetic pain syndrome (CRPS) by administering such prodrugs to patients in need of such treatment.

### FIELD OF THE DISCLOSURE

Disclosed are novel ketamine prodrugs, pharmaceutical preparations containing such prodrugs of ketamine and methods of treating, bipolar depression, neuropathic and chronic pain, including complex regional pain synthetic pain syndrome (CRPS) by administering such prodrugs to patients in need of such treatment.

### BACKGROUND OF THE INVENTION

It has recently been discovered that (2R,6R)-hydroxynorketamine is responsibile for ketamine's rapid-acting antidepressant effects (Nature, 2016). The authors report that the metabolism of (R,S)-ketamine to (2S,6S;2R,6R)-hydroxynorketamine (HNK) is essential for its antidepressant effects, and the (2R,6R)-HNK enantiomer lacks ketamine-related side effects but exerts rapid and sustained antidepressant actions in mice; these antidepressant effects are independent of NMDAR inhibition but require AMPAR activity

Metabolites and certain prodrugs are described in United States Patent Publication No. 2014/0296241 published Oct. 2, 2014 and issued as U.S. Pat. No. 9,650,352 on May 16, 2017 which is hereby incorporated by reference in its entirety.

### SUMMARY OF THE INVENTION

The present invention is directed to compounds of the Formula

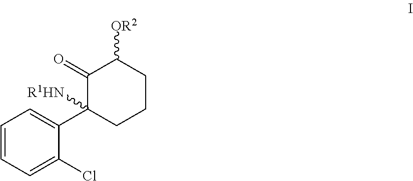

wherein R¹ is selected from hydrogen or a group of the formula

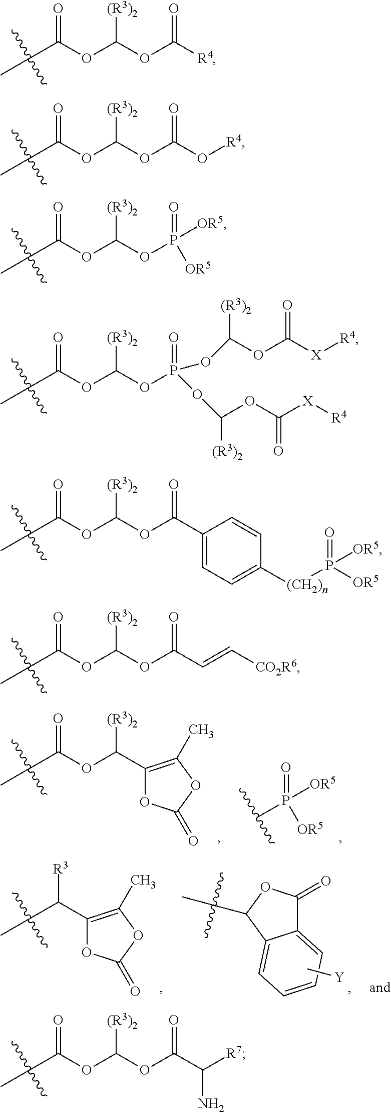

R² is selected from hydrogen or a group of the formula

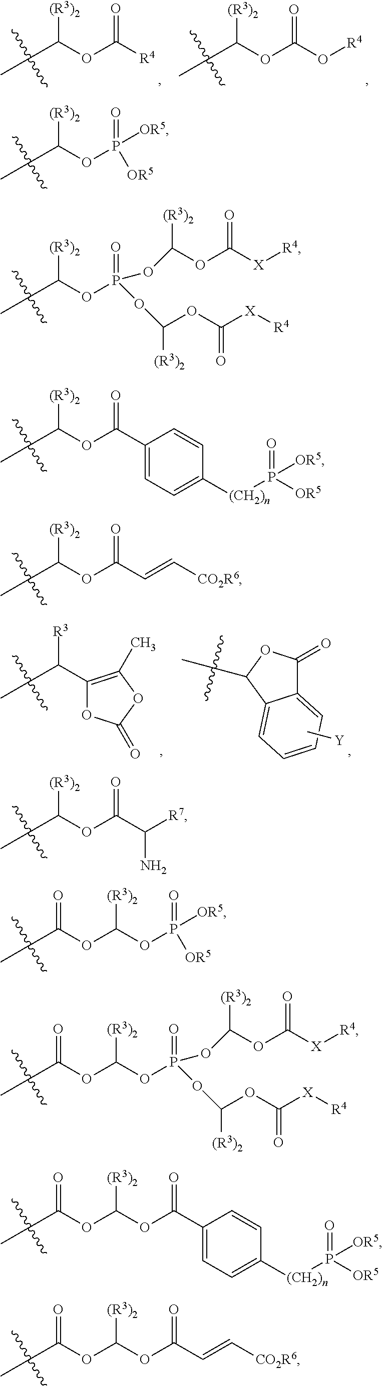

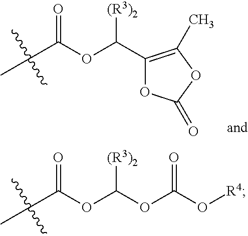

each R³ is independently H or CH₃;
each R⁴ is independently H, or alkyl;
each R⁵ is independently selected from the group consisting of H, alkyl, cycloalkyl, aryl, heteroaryl, heterocyclic and a salt selected from K, Na, Ca, Mg, primary (e.g., ethanolamine), secondary, and tertiary amines;
n is an integer selected from 0 or 1;
X is CH₂, CHR³, C(R³)₂, or O;
R⁶ is H, alkyl or a salt selected from K, Na, Ca, Mg, primary (e.g., ethanolamine), secondary, and tertiary amines;
R⁷ is an amino acid substituent; and
Y is H, alkyl, halogen, or CN.

As used herein, the term “alkyl” is defined to include saturated or unsaturated hydrocarbons including straight chains and branched chains and 1 to 20 carbon atoms. For example, as used herein, the term alkyl refers to linear or branched radicals (1 to 6 carbon atoms is a specific embodiment as are the long chain even numbered naturally occurring alkyl). Other embodiments include methyl, ethyl, n-propyl, isopropyl, n-butyl, iso-butyl, secondary-butyl, tertiary-butyl, hexyl, decanyl, and icosanyl). Alkyl groups may also optionally be substituted with from 1 to 5 suitable substituents. Unsaturated hydrocarbons have at least one carbon-carbon double bond, including straight chains and branched chains and 2 or more carbon atoms. For example, as used herein, the term alkyl includes straight or branched chain unsaturated radicals of 2 to 20 carbon atoms, including, but not limited to ethenyl, 1-propenyl, 2-propenyl (allyl), iso-propenyl, 2-methyl-1-propenyl, 1-butenyl, 2-butenyl, and the like; optionally substituted by 1 to 5 suitable substituents. When the compounds of Formulae Ia, Ib, Ic or Id, contain an alkenyl group, the alkenyl group may exist as the pure E (entgegen) form, the pure Z (zusammen) form, or any mixture thereof. Unsaturated hydrocarbons have at least one carbon-carbon triple bond, including straight chains and branched chains, and 2 to 20 carbon atoms. For example, as used herein, the term alkyl is used herein to include straight or branched hydrocarbon chain unsubstituted (e.g. alkynyl) radical having 2 to 20 carbon atoms and one triple bond; optionally substituted by 1 to 5 suitable substituents.

As used herein, the term “cycloalkyl” is defined to include saturated or unsaturated (non aromatic) monocyclic or bicyclic hydrocarbon rings (e.g., cyclopropyl, cyclobutyl, cyclopentyl, cyclohexyl, cycloheptyl, cyclooctyl, cyclononyl); optionally substituted by 1 to 5 suitable substituents. The cycloalkyl group has 3 to 12 carbon atoms. One group of monocyclic cycloalkyl rings have 3 to 6 carbon atoms. In another embodiment the cycloalkyl may optionally contain one, two or more non cumulative non aromatic double or triple bonds. Bicyclic hydrocarbon is defined to include a cycloalkyl as defined above which is bridged to a second carbocyclic ring (e.g., bicyclo[2.2.1]heptanyl, bicyclo[3.2.1]octanyl and bicyclo[5.2.0]nonanyl, etc.). Preferably, the bicycloalkyl group has 6 to 20 carbon atoms. More preferably, the bicycloalkyl group has 6 to 15 carbon atoms. Most preferably, the bicycloalkyl group has 6 to 12 carbon atoms. The bicycloalkyl is optionally substituted by 1 to 5 suitable substituents. In one embodiment the bicycloalkyl may optionally contain one, two or more non cumulative non aromatic double or triple bonds

As used herein, the term “aryl” is defined to include all-carbon monocyclic or fused-ring polycyclic (i.e., rings which share adjacent pairs of carbon atoms) groups having a completely conjugated pi-electron system. The aryl group has 6, 8, 9, 10 or 12 carbon atoms in the ring(s). In one embodiment the aryl group has 6 or 10 carbon atoms in the ring(s). One aryl group of particular interest is the 6 carbon atom phenyl ring. For example, as used herein, the term “aryl” means aromatic radicals containing from 6 to 10 carbon atoms such as phenyl, naphthyl, tetrahydronaphthyl, anthracenyl, indanyl and the like. The aryl group is optionally substituted by 1 to 5 suitable substituents.

As used herein, the term “heteroaryl” is defined to include monocyclic or fused-ring polycyclic aromatic heterocyclic groups with one or more heteroatoms selected from O, S and N in the ring. The heteroaryl group has 5 to 12 ring atoms including one to five heteroatoms selected from O, S, and N. For example, as used herein, the phrase “5 to 12 membered heteroaryl” means aromatic radicals containing at least one ring heteroatom selected from O, S and N and from 1 to 11 carbon atoms such as pyridyl, pyrazinyl, pyrimidinyl, pyridazinyl, thienyl, furyl, imidazolyl, pyrrolyl, oxazolyl (e.g., 1,3-oxazolyl, 1,2-oxazolyl), thiazolyl (e.g., 1,2-thiazolyl, 1,3-thiazolyl), pyrazolyl, tetrazolyl, triazolyl (e.g., 1,2,3-triazolyl, 1,2,4-triazolyl), oxadiazolyl (e.g., 1,2,3-oxadiazolyl), thiadiazolyl (e.g., 1,3,4-thiadiazolyl), quinolyl, isoquinolyl, benzothienyl, benzofuryl, indolyl, and the like. The heteroaryl group is optionally substituted by 1 to 5 suitable substituents.

As used herein, the term “heterocyclic” is defined to include a monocyclic, bridged, polycyclic or fused polycyclic saturated or unsaturated non-aromatic 3 to 13 membered ring including 1 or more heteroatoms selected from O, S and N. Examples of such heterocycloalkyl rings include azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydro-thiadiazinyl, morpholinyl, oxetanyl, tetrahydrodiazinyl, oxazinyl, oxathiazinyl, indolinyl, isoindolinyl, quinuclidinyl, chromanyl, isochromanyl, benzoxazinyl, and the like. Further examples of said heterocycloalkyl rings are tetrahydrofuran-2-yl, tetrahydrofuran-3-yl, imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperazin-1-yl, piperazin-2-yl, piperazin-3-yl, 1,3-oxazolidin-3-yl, isothiazolidine, 1,3-thiazolidin-3-yl, 1,2-pyrazolidin-2-yl, 1,3-pyrazolidin-1-yl, 1,2-tetrahydrothiazin-2-yl, 1,3-tetrahydrothiazin-3-yl, 1,2-tetrahydrodiazin-2-yl, 1,3-tetrahydrodiazin-1-yl, 1,4-oxazin-2-yl, 1,2,5-oxathiazin-4-yl and the like. The heterocycloalkyl ring is optionally substituted by 1 to 5 suitable substituents.

Amino Acid substituent as used herein refers to a group comprising an amine functionality and a carboxylic acid functionality. There are about 500 amino acids known. Amino Acids of particular interest include the 20 so called natural amino acids including Alanine, Arginine, Asparagine, Aspartic acid, Cysteine, Glutamic acid, Glutamine, Glycine, Ornithine, Proline, Selenocysteine, Serine, Tyrosine, Histidine, Isoleucine, Leucine, Lysine, Methionine, Phenylalanine, Threonine, Tryptophan, and Valine.

Amine salts as used herein and specifically primary, secondary or tertiary amines in groups such as R⁵ refers to a group of cation salts formed by reacting a primary, secondary or tertiary amine with a phosphonic acid precursor.

An “active agent” means any compound, element, or mixture that when administered to a patient alone or in combination with another agent confers, directly or indirectly, a physiological effect on the patient. When the active agent is a compound, salts, solvates (including hydrates) of the free compound or salt, crystalline and non-crystalline forms, as well as various polymorphs of the compound are included. Compounds may contain one or more asymmetric elements such as stereogenic centers, stereogenic axes and the like, e.g. asymmetric carbon atoms, so that the compounds can exist in different stereoisomeric forms. These compounds can be, for example, racemates or optically active forms.

“Administration” means dispensing a compound or composition containing the compound for use via any appropriate route, for example, oral administration, in either solid or liquid dosage form, inhalation, injection, suppository administration, or transdermal contact. “Administration” also include applying a compound or composition containing the compound via any appropriate route such as via oral administration, in either solid or liquid dosage form, inhalation, injection, suppository administration, or transdermal contact.

“Depressive symptoms” include low mood, diminished interest in activities, psychomotor slowing or agitation, changes in appetite, poor concentration or indecisiveness, excessive guilt or feelings of worthlessness, and suicidal ideations may occur in the context of depressive disorders, bipolar disorders, mood disorders due to a general medical condition, substance-induced mood disorders, other unspecified mood disorders, and also may be present in association with a range of other psychiatric disorders, including but not limited to psychotic disorders, cognitive disorders, eating disorders, anxiety disorders and personality disorders. The longitudinal course of the disorder, the history, and type of symptoms, and etiologic factors help distinguish the various forms of mood disorders from each other.

“Depression symptoms rating scale” refers to any one of a number of standardized questionnaires, clinical instruments, or symptom inventories utilized to measure symptoms and symptom severity in depression. Such rating scales are often used in clinical studies to define treatment outcomes, based on changes from the study's entry point(s) to endpoint(s). Such depression symptoms rating scales include, but are not limited to, The Quick Inventory of Depressive-Symptomatology Self-Report (QIDS-SR₁₆), the 17-Item Hamilton Rating Scale of Depression (HRSD₁₇), the 30-Item Inventory of Depressive Symptomatology (IDS-C₃₀), or The Montgomery-Asperg Depression Rating Scale (MADRS). Such ratings scales may involve patient self-report or be clinician rated. A 50% or greater reduction in a depression ratings scale score over the course of a clinical trial (starting point to endpoint) is typically considered a favorable response for most depression symptoms rating scales. “Remission” in clinical studies of depression often refers to achieving at, or below, a particular numerical rating score on a depression symptoms rating scale (for instance, less than or equal to 7 on the HRSD₁₇; or less than or equal to 5 on the QIDS-SR₁₆; or less than or equal to 10 on the MADRS).

A “patient” means any human or non-human animal in need of medical treatment. Medical treatment can include treatment of an existing condition, such as a disease or disorder, prophylactic or preventative treatment, or diagnostic treatment. In some embodiments the patient is a human patient.

“Pharmaceutical compositions” are compositions comprising at least one active agent, such as a compound or salt of Formula Ia-Id, and at least one other substance, such as a carrier, excipient, or diluent.

The term “carrier” applied to pharmaceutical compositions of the invention refers to a diluent, excipient, or vehicle with which an active compound is administered.

A “pharmaceutically acceptable excipient” means an excipient that is useful in preparing a pharmaceutical composition that is generally safe, non-toxic, and neither biologically nor otherwise undesirable, and includes an excipient that is acceptable for veterinary use as well as human pharmaceutical use. A “pharmaceutically acceptable excipient” as used in the present application includes both one and more than one such excipient.

The term “therapeutically effective amount” or “effective amount” means an amount effective, when administered to a human or non-human patient, to provide any therapeutic benefit. A therapeutic benefit may be an amelioration of symptoms, e.g., an amount effective to decrease the symptoms of a depressive disorder or pain. A therapeutically effective amount of a compound is also an amount sufficient to provide a significant positive effect on any indicia of a disease, disorder, or condition e.g. an amount sufficient to significantly reduce the frequency and severity of depressive symptoms or pain. A significant effect on an indicia of a disorder or condition includes a statistically significant in a standard parametric test of statistical significance such as Student's T-test, where p<0.05; though the effect need not be significant in some embodiments.

Another embodiment of the present invention relates to a compound of the formula

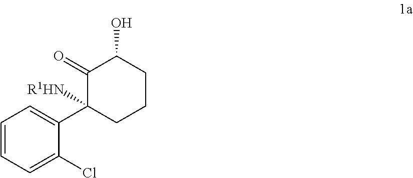

wherein R¹ is selected from hydrogen or a group of the formula

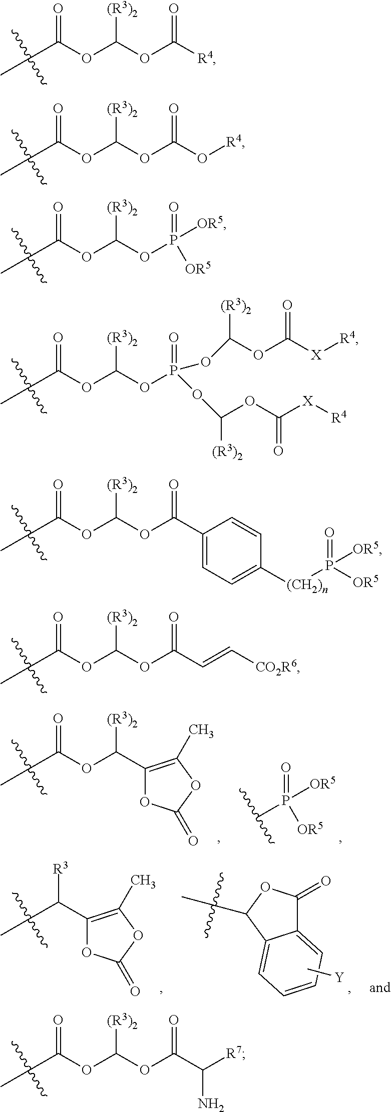

each R³ is independently H or CH₃;
each R⁴ is independently H, or alkyl;
each R⁵ is independently selected from the group consisting of H, alkyl, cycloalkyl, aryl, heteroaryl, heterocyclic and a salt selected from K, Na, Ca, Mg, primary (e.g., ethanolamine), secondary, and tertiary amines;
n is an integer selected from 0 or 1;
X is CH₂, CHR³, C(R³)₂, or O;
R⁶ is H, alkyl or a salt selected from K, Na, Ca, Mg, primary (e.g., ethanolamine), secondary, and tertiary amines;
R⁷ is an amino acid substituent; and
Y is H, alkyl, halogen, or CN.

Another embodiment of the present invention relates to a compound of the formula

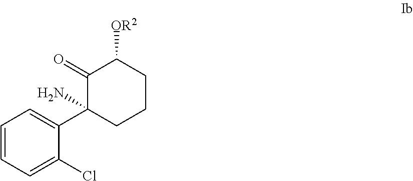

wherein R² is a group of the formula

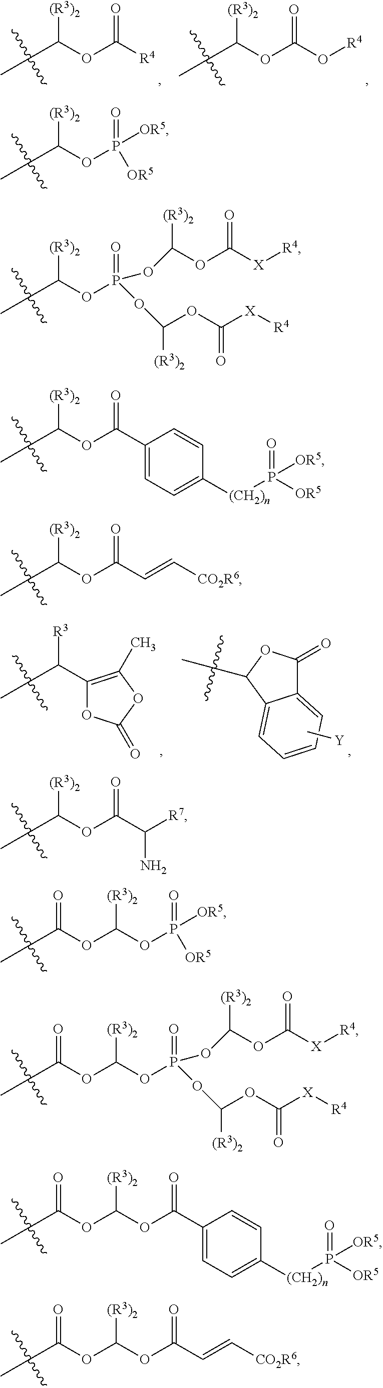

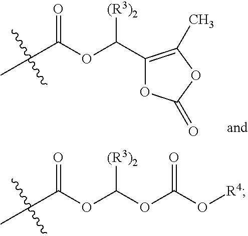

each R³ is independently H or CH₃;
each R⁴ is independently H or alkyl;
each R⁵ is independently selected from the group consisting of H, alkyl, cycloalkyl, aryl, heteroaryl, heterocyclic and a salt selected from K, Na, Ca, Mg, primary (e.g., ethanolamine), secondary, and tertiary amines;
n is an integer selected from 0 or 1;
X is CH₂, CHR³, C(R³)₂, or O;
R⁶ is H, alkyl or a salt selected from K, Na, Ca, Mg, primary (e.g., ethanolamine), secondary, and tertiary amines;
R⁷ is an amino acid substituent; and
Y is H, alkyl, halogen, or CN.

Another embodiment of the present invention relates to a compound of the formula

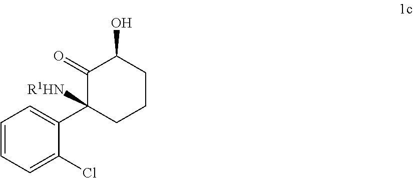

wherein R¹ is selected from hydrogen or a group of the formula

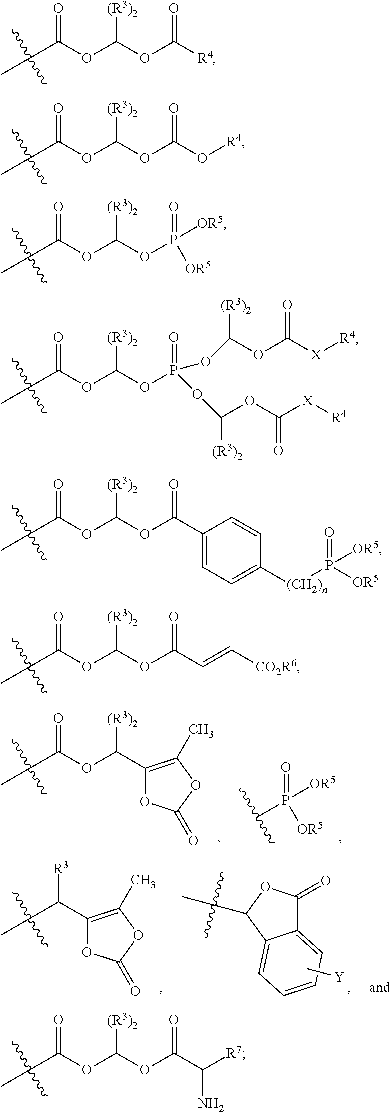

each R³ is independently H or CH₃;
each R⁴ is independently H, or alkyl;
each R⁵ is independently selected from the group consisting of H, alkyl, cycloalkyl, aryl, heteroaryl, heterocyclic and a salt selected from K, Na, Ca, Mg, primary (e.g., ethanolamine), secondary, and tertiary amines;
n is an integer selected from 0 or 1;
X is CH₂, CHR³, C(R³)₂, or O;
R⁶ is H, alkyl or a salt selected from K, Na, Ca, Mg, primary (e.g., ethanolamine), secondary, and tertiary amines;
R⁷ is an amino acid substituent; and
Y is H, alkyl, halogen, or CN.

Another embodiment of the present invention relates to a compound of the formula

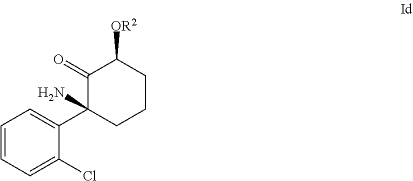

wherein R² is selected from hydrogen or a group of the formula

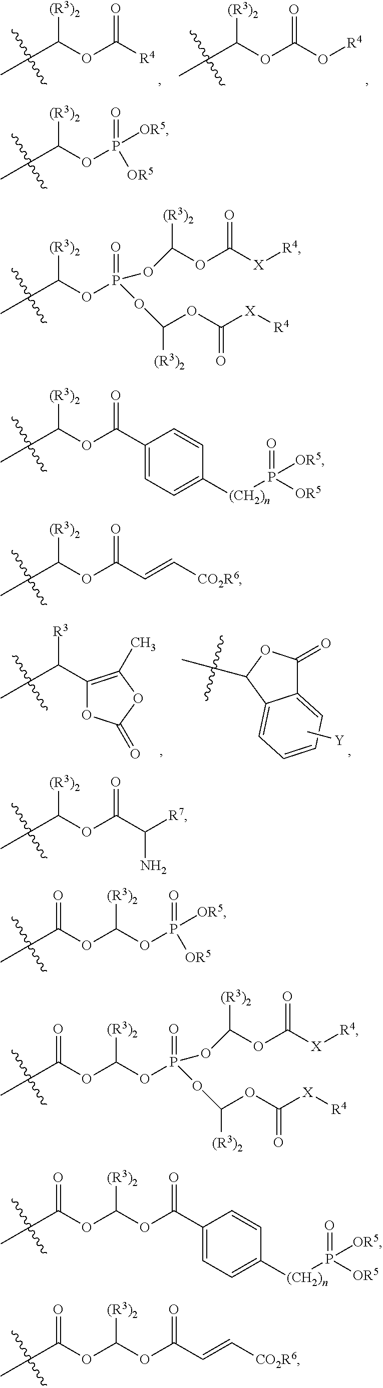

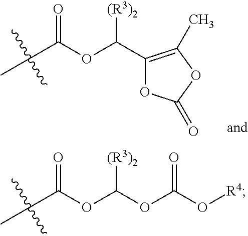

each R³ is independently H or CH₃;
each R⁴ is independently H or alkyl;
each R⁵ is independently selected from the group consisting of H, alkyl, cycloalkyl, aryl, heteroaryl, heterocyclic or a salt selected from K, Na, Ca, Mg, primary (e.g., ethanolamine), secondary, and tertiary amines;
n is an integer selected from 0 or 1;
X is CH₂, CHR³, C(R³)₂, or O;
R⁶ is H, alkyl or a salt selected from K, Na, Ca, Mg, Primary (e.g., ethanolamine), secondary, tertiary amines;
R⁷ is an amino acid substituent; and
Y is H, alkyl, halogen, or CN.

Another embodiment of the present invention relates to a compound of the Formula Ia or Ic wherein R¹ is a group of the formula

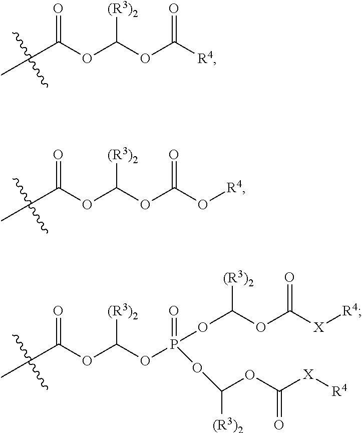

and wherein at least one R⁴ is hydrogen.

Another embodiment of the present invention relates to a compound of the aforesaid Formula Ia or Ic wherein at least one R⁴ is alkyl.

Another embodiment of the present invention relates to a compound of the Formula Ia or Ic wherein R¹ is a group of the formula

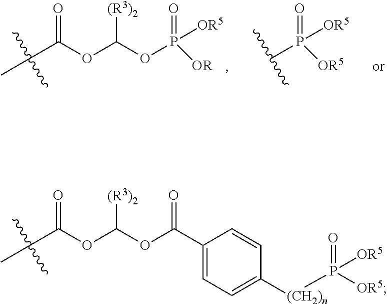

wherein at least one R⁵ is H.

Another embodiment of the present invention relates to a compound of the aforesaid formula Ia or Ic wherein at least one R⁵ is alkyl.

Another embodiment of the present invention relates to a compound of the aforesaid formula Ia or Ic wherein at least one R⁵ is cycloalkyl, aryl, heteroaryl or heterocyclic.

Another embodiment of the present invention relates to a compound of the aforesaid formula Ia or Ic wherein at least one R⁵ is a salt selected from K, Na, Ca and Mg.

Another embodiment of the present invention relates to a compound of the aforesaid formula Ia or Ic wherein at least one R⁵ is a primary (e.g., ethanolamine), secondary or tertiary amine salt.

Another embodiment of the present invention relates to a compound of the Formula Ia or Ic wherein including each of the aforesaid R², R⁴ and R⁵ groups wherein at least one R³ is H.

Another embodiment of the present invention relates to a compound of the Formula Ia or Ic wherein including each of the aforesaid R², R⁴ and R⁵ groups wherein at least one R³ is CH₃.

Another embodiment of the present invention relates to a compound of the Formula Ib or Id wherein R² is a group of the formula

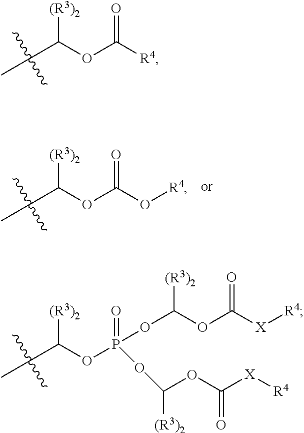

and wherein at least one R⁴ is hydrogen.

Another embodiment of the present invention relates to a compound of the aforesaid Formula Ib or Id wherein at least one R⁴ is alkyl.

Another embodiment of the present invention relates to a compound of the Formula Ib or Id wherein R² is a group of the formula

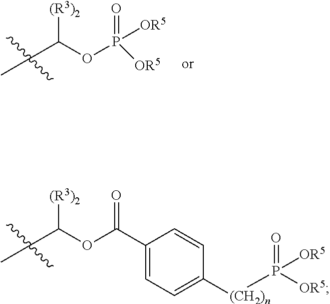

wherein at least one R⁵ is H.

Another embodiment of the present invention relates to a compound of the aforesaid formula Ib or Id wherein at least one R⁵ is alkyl.

Another embodiment of the present invention relates to a compound of the aforesaid formula Ib or Id wherein at least one R⁵ is cycloalkyl, aryl, heteroaryl or heterocyclic.

Another embodiment of the present invention relates to a compound of the aforesaid formula Ib or Id wherein at least one R⁵ is a salt selected from K, Na, Ca and Mg.

Another embodiment of the present invention relates to a compound of the aforesaid formula Ib or Id wherein at least one R⁵ is a primary (e.g., ethanolamine), secondary or tertiary amine salt.

Another embodiment of the present invention relates to a compound of the Formula Ib or Id wherein including each of the aforesaid R², R⁴ and R⁵ groups wherein at least one R³ is H.

Another embodiment of the present invention relates to a compound of the Formula Ib or Id wherein including each of the aforesaid R², R⁴ and R⁵ groups wherein at least one R³ is CH₃.

Another embodiment of the present invention relates to the compound

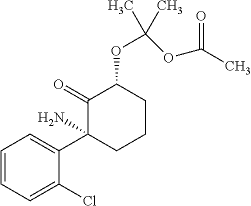 [SMILES: CC(=O)OC(C)(C)O[C@@H]1CCC[C@@](N)(c2ccccc2Cl)C1=O]

(((1R,3R)-3-amino-3-(2-chlorophenyl)-2-oxocyclohexyl)oxy)methyl pivalate. Another embodiment of the present invention relates to the compound

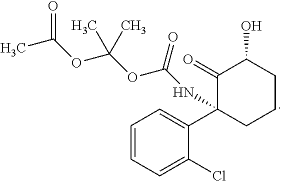 [SMILES: CC(=O)OC(C)(C)OC(=O)N[C@@]1(c2ccccc2Cl)CCC[C@@H](O)C1=O]

Another embodiment of the present invention relates to the compound

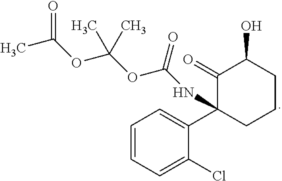 [SMILES: CC(=O)OC(C)(C)OC(=O)N[C@]1(c2ccccc2Cl)CCC[C@H](O)C1=O]

Another embodiment of the present invention relates to the compound

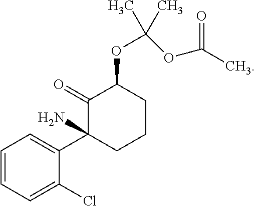 [SMILES: CC(=O)OC(C)(C)O[C@H]1CCC[C@](N)(c2ccccc2Cl)C1=O]

Another embodiment of the present invention relates to the compound

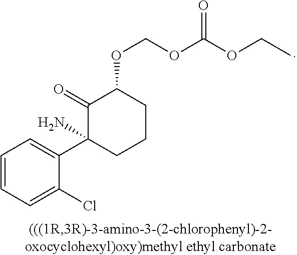 [SMILES: CCOC(=O)OCO[C@@H]1CCC[C@@](N)(c2ccccc2Cl)C1=O]

Another embodiment of the present invention relates to the compound

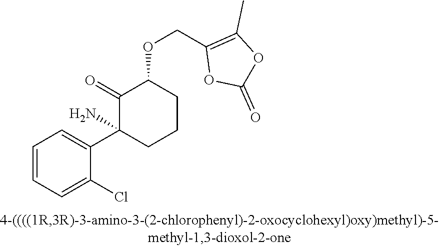 [SMILES: Cc1oc(=O)oc1CO[C@@H]1CCC[C@@](N)(c2ccccc2Cl)C1=O]

Compounds disclosed herein can be administered as the neat chemical, but are preferably administered as a pharmaceutical composition. Accordingly, the disclosure provides pharmaceutical compositions comprising a compound or pharmaceutically acceptable salt of a compound of Formula I or Ia-Id, together with at least one pharmaceutically acceptable carrier. The pharmaceutical composition may contain a compound Formula I or Ia-Id as the only active agent, but is preferably contains at least one additional active agent. In certain embodiments the pharmaceutical composition is an oral dosage form that contains from about 0.1 mg to about 1000 mg, from about 1 mg to about 500 mg, or from about 10 mg to about 200 mg of a compound of Formula I and optionally from about 0.1 mg to about 2000 mg, from about 10 mg to about 1000 mg, from about 100 mg to about 800 mg, or from about 200 mg to about 600 mg of an additional active agent in a unit dosage form.

Compounds disclosed herein may be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present invention.

The pharmaceutical compositions can be formulated for oral administration. Preferred oral dosage forms are formulated for once a day or twice a day administration. These compositions contain between 0.1 and 99 weight % (wt. %) of a compound of Formula I and usually at least about 5 wt. % of a compound of Formula. Some embodiments contain from about 25 wt. % to about 50 wt. % or from about 5 wt. % to about 75 wt. % of the compound of Formula I or Ia-Id.

A compound of Formula I or Ia-Id is effective in the treatment of neuropathic and chronic pain, including the treatment of patients suffering from complex regional pain syndrome (CRPS).

The disclosure includes a methods of treating bipolar depression, major depressive disorder, schizophrenia, Alzheimer's dementia, amyotrophic lateral sclerosis, complex regional pain syndrome (CRPS), chronic pain, or neuropathic pain comprising administering a pharmaceutical composition containing an effective amount of a compound of Formula I or Ia-Id, together with a pharmaceutically acceptable carrier to a patient in need of such treatment.

Methods of treatment include providing certain dosage amounts of a compound of Formula I or Ia-Id to a patient. Dosage levels of each active agent of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single unit dosage form will vary depending upon the patient treated and the particular mode of administration.

In certain embodiments a therapeutically effect amount is an amount that provide a plasma Cmax of a compound of Formula I or Ia-Id of about of 0.25 mcg/mL to about 125 mcg/mL, or about 1 mcg/mL to about 50 mcg/mL. For peripheral indications formulations and methods that provide a Cmax of about 0.25 mcg/mL to about 25 mcg/mL are preferred, while for CNS indications, formulations and methods that provide a plasma Cmax of about 0.25 mcg/mL to about 125 mcg/mL are preferred. The disclosure also includes IV pharmaceutical compositions that provide about 0.2 mg to about 500 mg per dose of a compound of Formula I, for peripheral indications compounds that provide about 0.5 mg to about 500 mg/dose are preferred.

In another aspect the disclosure provides a method of treating bipolar depression, major depressive disorder, schizophrenia, Alzheimer's dementia, amyotrophic lateral sclerosis, complex regional pain syndrome (CRPS), chronic pain, or neuropathic pain comprising administering a pharmaceutical composition containing an effective amount of a compound of Formula I or Ia-Id or a pharmaceutically acceptable salt thereof, together with a pharmaceutically acceptable carrier to a patient in need of such treatment.

The disclosure also includes a method of treating bipolar depression, schizophrenia, Alzheimer's dementia, amyotrophic lateral sclerosis, complex regional pain syndrome (CRPS), chronic pain, migraine, or neuropathic pain comprising administering an effective amount of isolated hydroxynorketamine diastereomers, such as (2R,6R)-hydroxynorketamine, (2S,6S)-hydroxynorketamine or isolated (R)- or (S)-dehydronorketamine to a patient in need of such treatment.

In yet another aspect the disclosure includes a method of treating a patient for bipolar depression, complex regional pain syndrome (CRPS), chronic pain, or neuropathic pain comprising
(1) determining that the patient is a ketamine non-responder; and
(2) administering an effective amount of Formula I or Ia-Id to the patient.

Applicants have determined that certain compounds of Formula I are potent serine racemase inhibitors. The disclosure also provides a method of inhibiting serine racemase comprising contacting cells with a concentration of a compound of Formula I or Ia-Id sufficient to inhibit serine racemase in vitro.

### DETAILED DESCRIPTION

Compounds of Formula I, Ia, Ib, Ic and Id may be prepared by reacting a compound of Formula I wherein R¹ is hydrogen or methyl and R² is hydrogen by methods well known to those skilled in the art. For instance, certain methods are described in United States Patent Publication No. 2014/0296241 published Oct. 2, 2014 and issued as U.S. Pat. No. 9,650,352 on May 16, 2017 which is hereby incorporated by reference in its entirety.

The compound of Formula I, Ia, Ib, Ic and Id may be the only active agent administered or may be administered together with an additional active agent. For example the compound of Formula I, Ia, Ib, Ic or Id may administered together with another active agent that is chosen from any of the following: Antidepressants: escitalopram, fluoxetine, paroxetine, duloxetine, sertraline, citalopram, bupropion, venlafaxine, duloxetine, naltrexone, mirtazapine, venlafaxine, atomoxetine, bupropion, doxepin, amitriptyline, clomipramine, nortriptyline, buspirone, aripiprazole, clozapine, loxapine, olanzapine, quetiapine, risperidone, ziprasidone, carbamazepine, gabapentin, lamotrigine, phenyloin, pregabalin, donepezil, galantamine, memantine, rivastigmine, tramiprosate, or pharmaceutically active salts or prodrugs thereof, or a combination of the foregoing; Schizophrenia Medications: aripiprazole, lurasidone, asenapine, clozapine, ziprasidone, risperidone, quetiapine, stelazine, olanzapine, loxapine, flupentioxol, perphenazine, haloperidol, chlorpromazine, fluphenazine, prolixin, paliperidone; Alzheimer's Dementia Medications: donepezil, rivastigmine, galantamine, memantine ALS Medications: riluzole Pain Medications: acetaminophen, aspirin, NSAIDS, including Diclofenac, Diflunisal, Etodolac, Fenoprofen, Flurbiprofen, Ibuprofen, Indomethacin, Ketoprofen, Ketorolac, Meclofenamate, Mefenamic Acid, Meloxicam, Nabumetone, Naproxen, Oxaprozin, Phenylbutazone, Piroxicam, Sulindac, Tolmetinopiods, Cox-2 inhibitors such as celcoxib, and narcotic pain medications such as Buprenorphine, Butorphanol, Codeine, Hydrocodone, Hydromorphone, Levorphanol, Meperidine, Methadone, Morphine, Nalbuphine, Oxycodone, Oxymorphone, Pentazocine, Propoxyphene, the central analgesic tramadol.

The preceding list of additional active agents is meant to be exemplary rather than fully inclusive. Additional active agents not included in the above list may be administered in combination with a compound of Formula I, Ia, Ib, Ic and Id. The additional active agent will be dosed according to its approved prescribing information, though in some embodiments the additional active agent will be dosed at less the typically prescribed dose and in some instances less than the minimum approved dose.

The disclosure includes a method of treating bipolar depression and major depressive disorder where an effective amount of the compound is an amount effective to decrease depressive symptoms, wherein a decrease in depressive symptoms is the achievement of a 50% or greater reduction of symptoms identified on a depression symptom rating scale, or a score less than or equal to 7 on the HRSD17, or less than or equal to 5 on the QID-SR16, or less than or equal to 10 on the MADRS.

The disclosure provides an amount effective to decrease painful symptoms; wherein a decrease in painful symptom is the achievement of a 50% or greater reduction of painful symptoms on a pain rating scale.

Methods such as Serine Racemase Inhibition Assays, Cell Lines and Cell Cultures, CE-LIF (Capillary Electrophoresis-Laser Induced Fluorescence) Analysis, and Effect of Compounds on Currents Evoked by Acetylcholine Receptors are as described in in United States Patent Publication No. 2014/0296241 published Oct. 2, 2014 and issued as U.S. Pat. No. 9,650,352 on May 16, 2017. Additional description is found in “Ketamine Mechanism of Action: Separating the Wheat from the Chaff,” Panos Zanos et. al., Neuropsychopharmacology 42, 368-369 (January 2017), doi:10.1038/npp.2016.210.

1. A compound of the formula 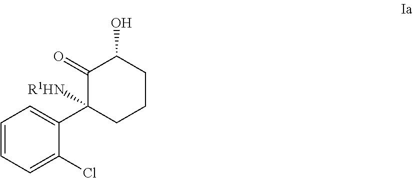
wherein R¹ is selected from hydrogen or a group of the formula 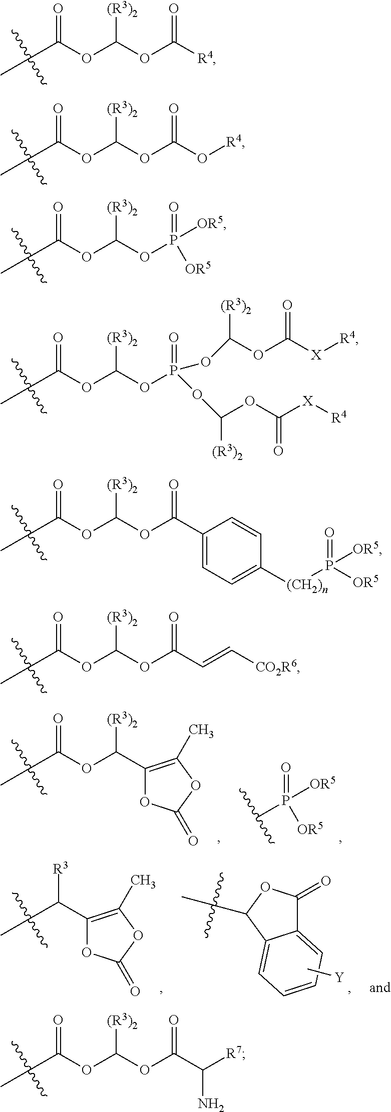
each R³ is independently H or CH₃;
each R⁴ is independently H or alkyl;
each R⁵ is independently selected from the group consisting of H, alkyl, aryl, cycloalkyl, heteroaryl, heterocyclic and a salt selected from K, Na, Ca, Mg, primary, secondary, and tertiary amines;
n is an integer selected from 0 or 1;
X is CH₂, CHR³, C(R³)₂, or O;
R⁶ is H, alkyl or a salt selected from K, Na, Ca, Mg, primary (e.g., ethanolamine), secondary, and tertiary amines;
R⁷ is an amino acid substituent; and
Y is H, alkyl, halogen, or CN.

2. A compound of the formula 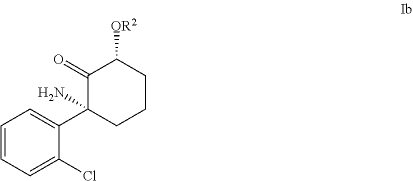
wherein R² is selected from hydrogen or a group of the formula 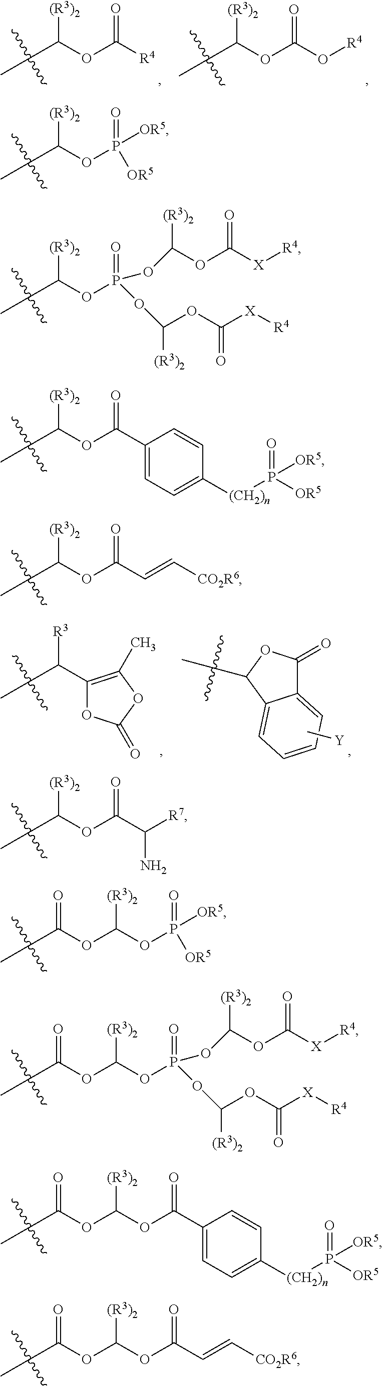 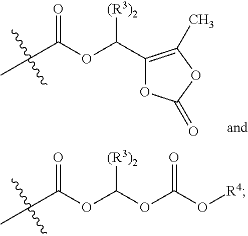
each R³ is independently H or CH₃;
each R⁴ is independently H or alkyl;
each R⁵ is independently selected from the group consisting of H, alkyl, cycloalkyl, aryl, heteroaryl, heterocyclic and a salt selected from K, Na, Ca, Mg, primary, secondary, and tertiary amines;
n is an integer selected from 0 or 1;
X is CH₂, CHR³, C(R³)₂, or O;
R⁶ is H, alkyl or a salt selected from K, Na, Ca, Mg, primary, secondary, and tertiary amines;
R⁷ is an amino acid substituent; and
Y is H, alkyl, halogen, or CN.

3. A compound of the formula 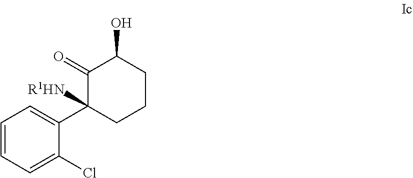
wherein R¹ is selected from hydrogen or a group of the formula 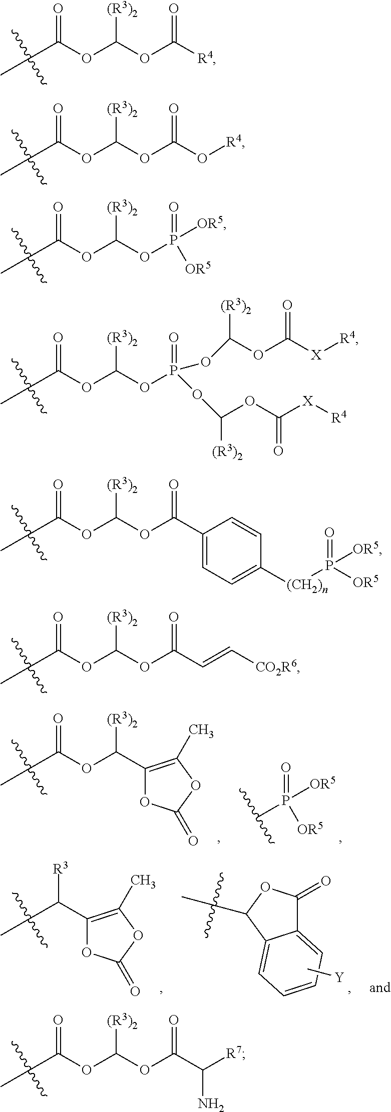
each R³ is independently H or CH₃;
each R⁴ is independently H, or alkyl;
each R⁵ is independently selected from the group consisting of H, alkyl, cycloalkyl, aryl, heteroaryl, heterocyclic and a salt selected from K, Na, Ca, Mg, primary, secondary, and tertiary amines;
n is an integer selected from 0 or 1;
X is CH₂, CHR³, C(R³)₂, or O;
R⁶ is H, alkyl or a salt selected from K, Na, Ca, Mg, primary, secondary, and tertiary amines;
R⁷ is an amino acid substituent; and
Y is H, alkyl, halogen, or CN.

4. A compound of the formula 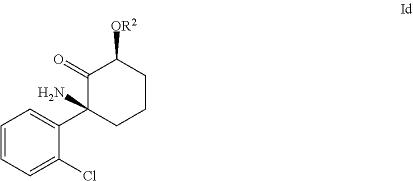
wherein R² is selected from hydrogen or a group of the formula 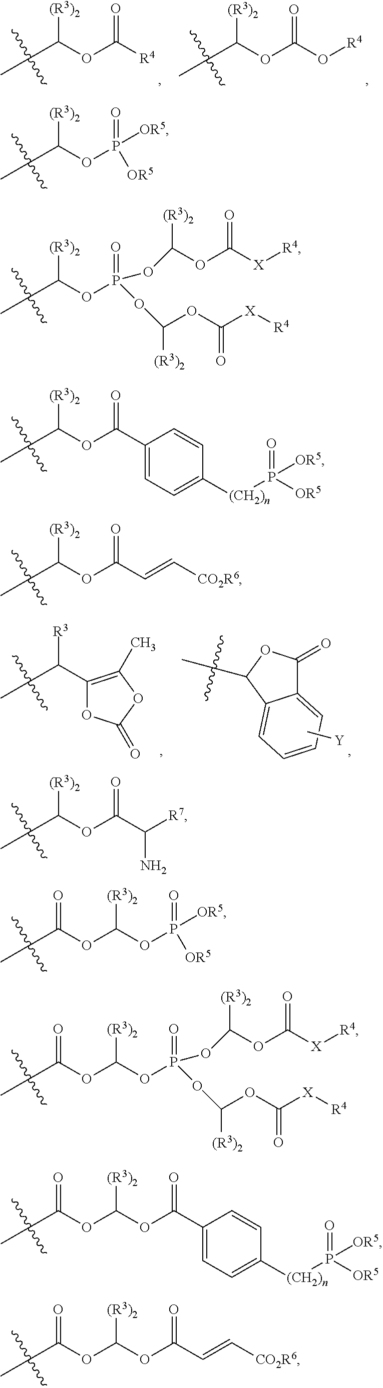 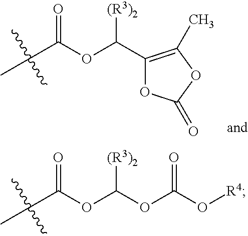
each R³ is independently H or CH₃;
each R⁴ is independently H or alkyl;
each R⁵ is independently selected from the group consisting of H, alkyl, cycloalkyl, heteroaryl, heterocyclic or a salt selected from K, Na, Ca, Mg, primary, secondary, and tertiary amines;
n is an integer selected from 0 or 1;
X is CH₂, CHR³, C(R³)₂, or O;
R⁶ is H, alkyl or a salt selected from K, Na, Ca, Mg, Primary, secondary, tertiary amines;
R⁷ is an amino acid substituent; and
Y is H, alkyl, halogen, or CN.

5. A method of treating bipolar depression, major depressive disorder, schizophrenia, Alzheimer's dementia, amyotrophic lateral sclerosis, complex regional pain syndrome (CRPS), chronic pain, migraine, or neuropathic pain comprising administering an effective amount of a compound of Formula I or a pharmaceutically acceptable salt thereof, together with a pharmaceutically acceptable carrier to a patient in need of such treatment.

6. The method of claim 5 wherein an effective amount is an amount effective to decrease painful symptoms; wherein a decrease in painful symptom is the achievement of a 50% or greater reduction of painful symptoms on a pain rating scale.
